(19) United States
(12) Patent Application Publication
(10) Pub. No.: US 2025/0290569 A1
(43) Pub. Date: Sep. 18, 2025

(54) Actuator Actuated Valves for Maintaining Multi-Lumen Patency and Method for Pulsatile Flow Lumen Flushing

(71) Applicants: Becton, Dickinson and Company, Franklin Lakes, NJ (US); Ferrosan Medical Devices SP. Z O. O., Szczecin (PL)

(72) Inventors: Kevin M. Ryan, Whitehouse Station, NJ (US); Angela Marie Huenerfauth, Morris Plains, NJ (US); Frederik Sonnenborg, Jyllinge (DK); Søren Falkesgaard Ørts, Virum (DK); Søren Christensen, Allerød (DK)

(21) Appl. No.: 18/861,228

(22) PCT Filed: May 4, 2023
(86) PCT No.: PCT/US2023/020909

§ 371 Date: Oct. 29, 2024

Related U.S. Application Data
(60) Provisional application No. 63/338,677, filed on May 5, 2022.

(51) Int. Cl. F16K 11/085; A61M 5/14; A61M 5/168

(52) U.S. Cl. CPC F16K 11/0856; A61M 5/16881; A61M 2005/1403

(57) ABSTRACT

An actuator actuated diverter valve for maintaining multi-lumen patency may include a valve manifold, a valve spool, and a rotary actuator. The valve manifold may include an input port, a first output port, and a second output port. The valve spool may extend within the valve manifold. The valve spool may define a first valve passageway and a second valve passageway, and the valve spool may be rotatable relative to the valve manifold between a first position in which the input port and the first output port are in fluid communication via the first valve passageway and a second position in which the input port and the second output port are in fluid communication via the second valve passageway. The rotary actuator may be configured to rotate the valve spool relative to the valve manifold between the first position and the second position.

### CROSS-REFERENCE TO RELATED APPLICATION

The present application claims priority to U.S. Provisional Application Ser. No. 63/338,677, entitled “Actuator Actuated Valves for Maintaining Multi-Lumen Patency and Method for Pulsatile Flow Lumen Flushing”, filed May 5, 2022, the entire disclosure of which is hereby incorporated by reference in its entirety.

### BACKGROUND

### 1. Field

The present disclosure relates generally to a fluid delivery and, in some non-limiting embodiments or aspects, to actuator actuated valves for maintaining multi-lumen patency and/or a method for pulsatile flow IV line flushing.

### 2. Technical Considerations

There is an increasing clinical need for patients to have dual or triple lumen IV catheters. These multi-lumen catheters are used to deliver multiple IV medication infusions to a patient at the same time in situations where the medications cannot be mixed together until the medications are in the blood stream of the patient. These multi-lumen IV catheters are often kept in-dwelling in the patient for weeks or months. During this in-dwelling time, the multiple medication infusions may only be delivered periodically for relatively short durations. For the remainder of the in-dwelling time, only a single IV medication infusion (or no medication infusion) may be delivered. However, for these times when only a single medication infusion (or no medication infusion) is being delivered, there is a challenge in keeping the unused catheter lumens patent (e.g., open, etc.) because catheter lines may tend to occlude (e.g., become block, etc.) due to various biological responses in the patient over long periods of time between active flow in the lumens. Existing solutions for this challenge are not ideal because a clinician has to flush the unused lumen(s) with saline and/or multiple IV drip bags, even if the patient is already receiving the one IV medication infusion.

### SUMMARY

Accordingly, provided are improved systems, devices, products, apparatus, and/or methods for maintaining multi-lumen patency and/or pulsatile flow lumen flushing.

According to some non-limiting embodiments or aspects, provided is an actuator actuated valve for maintaining multi-lumen patency, including: a valve manifold including an input port, a first output port, and a second output port; and a valve spool extending within the valve manifold, wherein the valve spool defines a first valve passageway and a second valve passageway, wherein the valve spool is rotatable relative to the valve manifold between a first position in which the input port and the first output port are in fluid communication via the first valve passageway and a second position in which the input port and the second output port are in fluid communication via the second valve passageway.

In some non-limiting embodiments or aspects, when the valve spool is between the first position and the second position, the input port is not in fluid communication with the first output port and the second output port.

In some non-limiting embodiments or aspects, when the valve spool is between the first position and the second position, the input port is in fluid communication with each of the first output port and the second output port.

In some non-limiting embodiments or aspects, the valve spool is rotatable relative to the valve manifold between the first position, the second position, and a third position in which the input port is not in fluid communication with the first output port and the second output port.

In some non-limiting embodiments or aspects, when the valve spool is in the first position, the input port is not in fluid communication with the second output port, and wherein, when the valve spool is in the second position, the input port is not in fluid communication with the first output port.

In some non-limiting embodiments or aspects, the valve manifold further includes at least one further output port, wherein the valve spool further defines at least one further valve passageway, wherein the valve spool is further rotatable relative to the valve manifold between the first position, the second position, and at least one further position in which in the input port and the at least one further output port are in fluid communication via the at least one further valve passageway.

In some non-limiting embodiments or aspects, the valve spool is rotatable between a plurality of positions including the first position, the second position, and the at least one further position, and wherein, in the plurality of positions, (i) a single output port of the first output port, the second output port, and the at least one further output port is in fluid communication with the input port via a single valve passageway of the first valve passageway, the second valve passageway, and the at least one further valve passageway and (ii) the other output ports of the first output port, the second output port, and the at least one further output port are not in fluid communication with the input port via the other valve passageways of the first valve passageway, the second valve passageway, and the at least one further valve passageway.

In some non-limiting embodiments or aspects, the valve further includes: a rotary actuator configured to rotate the valve spool relative to the valve manifold between the first position and the second position.

In some non-limiting embodiments or aspects, the valve further includes: a communication device configured to receive control data from an external computing device; and a processor configured to control, based on the control data, the rotary actuator to rotate the valve spool relative to the valve manifold between the first position and the second position.

In some non-limiting embodiments or aspects, the communication device includes a wireless communication device configured to wirelessly receive the control data from the external device.

In some non-limiting embodiments or aspects, the valve further includes: a housing connected to the valve spool, wherein the housing includes the rotary actuator, the communication device, and the processor.

In some non-limiting embodiments or aspects, the housing further includes a memory, a power source, and a user feedback device.

In some non-limiting embodiments or aspects, the valve further includes: a first check valve integrated with the first output port; a second check valve integrated with the second output port.

In some non-limiting embodiments or aspects, the input port is removably connected to a fluid output of a fluid reservoir.

In some non-limiting embodiments or aspects, the input port is integrated with a fluid output of a fluid reservoir.

In some non-limiting embodiments or aspects, the fluid reservoir includes a syringe, and wherein the input port is integrated with a tip of the syringe.

In some non-limiting embodiments or aspects, the valve spool includes an actuator interface extending from an end of the valve spool, and wherein the actuator interface is configured to interface with a rotary actuator.

According to some non-limiting embodiments or aspects, provided is a system for maintaining multi-lumen patency, including: an actuator actuated diverter valve including: a valve manifold including an input port, a first output port, and a second output port; and a valve spool extending within the valve manifold, wherein the valve spool defines a first valve passageway and a second valve passageway, wherein the valve spool is rotatable relative to the valve manifold between a first position in which the input port and the first output port are in fluid communication via the first valve passageway and a second position in which the input port and the second output port are in fluid communication via the second valve passageway; and a rotary actuator configured to rotate the valve spool relative to the valve manifold between the first position and the second position; a multi-lumen catheter including: a first lumen connected to the first output port; and a second lumen connected to the second output port; and a fluid reservoir connected to the input port.

In some non-limiting embodiments or aspects, the fluid reservoir includes one of an IV bag, an infusion pump, and a syringe.

In some non-limiting embodiments or aspects, the system further includes: a communication device configured to receive control data from an external computing device; and a processor configured to control, based on the control data, the rotary actuator to rotate the valve spool relative to the valve manifold between the first position and the second position.

In some non-limiting embodiments or aspects, the valve manifold further includes at least one further output port, wherein the valve spool further defines at least one further valve passageway, wherein the valve spool is further rotatable relative to the valve manifold between the first position, the second position, and at least one further position in which in the input port and the at least one further output port are in fluid communication via the at least one further valve passageway.

In some non-limiting embodiments or aspects, the valve spool is rotatable between a plurality of positions including the first position, the second position, and the at least one further position, and wherein, in the plurality of positions, (i) a single output port of the first output port, the second output port, and the at least one further output port is in fluid communication with the input port via a single valve passageway of the first valve passageway, the second valve passageway, and the at least one further valve passageway and (ii) the other output ports of the first output port, the second output port, and the at least one further output port are not in fluid communication with the input port via the other valve passageways of the first valve passageway, the second valve passageway, and the at least one further valve passageway.

In some non-limiting embodiments or aspects, the system further includes: an infusion pump including the rotary actuator, wherein the valve spool includes an actuator interface extending from an end of the valve spool that interfaces with the rotary actuator.

According to some non-limiting embodiments or aspects, provided is a method for pulsatile flow IV line flushing, including: (i) measuring, with an infusion pump, a baseline fluid pressure of an IV catheter line connected to the infusion pump and a patient, wherein the baseline fluid pressure is measured when a flow rate of a fluid through the IV catheter line is zero; (ii) increasing, with the infusion pump, the flow rate of the fluid through the IV catheter line until a current pressure of the IV catheter line satisfies a predetermined delta pressure setting above the measured baseline fluid pressure; (iii) in response to the current pressure of the IV catheter line satisfying the predetermined delta pressure setting above the measured baseline fluid pressure, controlling, with the infusion pump, based on the current pressure of the IV catheter line, the flow rate of the fluid through the IV catheter line to maintain the current pressure of the IV catheter line at the predetermined delta pressure setting above the baseline fluid pressure until a predetermined pulsatile flow volume is infused through the IV catheter line; (iv) in response to the predetermined pulsatile flow volume being infused into the IV catheter line, decreasing, with the infusion pump, the flow rate of the fluid through the IV catheter line until the flow rate is zero; (v) in response to decreasing the flow rate of the fluid through the IV catheter line to zero, maintaining, with the infusion pump, the flow rate of the fluid through the IV catheter line at zero for a predetermined period of time; and (vi) after the predetermined period of time, repeating steps (ii)-(v) until a predetermined total flush volume is infused through the IV catheter line.

In some non-limiting embodiments or aspects, if the measured baseline fluid pressure is measured as greater than a threshold baseline fluid pressure, a value of the measured baseline fluid pressure is set as the threshold baseline fluid pressure for determining the predetermined delta pressure setting above the measured baseline fluid pressure.

In some non-limiting embodiments or aspects, the infusion pump linearly increases the flow rate of the fluid through the IV catheter line until the current pressure of the IV catheter line satisfies the predetermined delta pressure setting above the measured baseline fluid pressure.

In some non-limiting embodiments or aspects, the infusion pump controls the flow rate of the fluid through the IV catheter line to remain at or below a safety threshold flow rate.

In some non-limiting embodiments or aspects, the predetermined period of time is 5 seconds, and wherein the predetermined total flush volume is in a range of 5 mL to 10 ml.

According to some non-limiting embodiments or aspects, provided is an infusion pump, including: a pressure sensor configured to measure a baseline fluid pressure of an IV catheter line connected to the infusion pump and a patient, wherein the baseline fluid pressure is measured when a flow rate of a fluid through the IV catheter line is zero; a fluid pump; and at least one processor programmed and/or configured to: (i) control the fluid pump to increase the flow rate of the fluid through the IV catheter line until a current pressure of the IV catheter line satisfies a predetermined delta pressure setting above the measured baseline fluid pressure; (ii) in response to the current pressure of the IV catheter line satisfying the predetermined delta pressure setting above the measured baseline fluid pressure, control, based on the current pressure of the IV catheter line, the fluid pump to control the flow rate of the fluid through the IV catheter line to maintain the current pressure of the IV catheter line at the predetermined delta pressure setting above the baseline fluid pressure until a predetermined pulsatile flow volume is infused through the IV catheter line; (iii) in response to the predetermined pulsatile flow volume being infused into the IV catheter line, control the fluid pump to decrease the flow rate of the fluid through the IV catheter line until the flow rate is zero; (iv) in response to decreasing the flow rate of the fluid through the IV catheter line to zero, control the fluid pump to maintain the flow rate of the fluid through the IV catheter line at zero for a predetermined period of time; and (i) after the predetermined period of time, repeat steps (i)-(iv) until a predetermined total flush volume is infused through the IV catheter line.

In some non-limiting embodiments or aspects, the flow rate of the fluid through the IV catheter line is linearly increased until the current pressure of the IV catheter line satisfies the predetermined delta pressure setting above the measured baseline fluid pressure.

In some non-limiting embodiments or aspects, the flow rate of the fluid through the IV catheter line is controlled to remain at or below a safety threshold flow rate.

In some non-limiting embodiments or aspects, the predetermined period of time is 5 seconds, and wherein the predetermined total flush volume is in a range of 5 mL to 10 ml.

According to some non-limiting embodiments or aspects, provided is an actuator actuated valve for maintaining multi-lumen patency, comprising: a valve body including a plurality of valve passageways extending between a plurality of input ports and a plurality of output ports, wherein the plurality of input ports is configured to be connected to a plurality of fluid outputs of a fluid reservoir; and a plurality of flexible membranes defining a portion of the plurality of valve passageways, wherein each flexible membrane of the plurality of flexible membranes (i) includes a first face that faces a valve passageway of the plurality of valve passageways and a second face opposite the first face and (ii) is configured for movement with respect to the valve passageway in response to a force applied to the second face of that flexible membrane to form a barrier against a flow of fluid in the valve passageway.

In some non-limiting embodiments or aspects, the valve further includes: a linear actuator configured to apply the force to the second face of each flexible membrane to form the barrier against the flow of fluid in the valve passageway.

In some non-limiting embodiments or aspects, the valve further includes: a communication device configured to receive control data from an external computing device; and a processor configured to control, based on the control data, the linear actuator between a plurality of positions in which the force is applied to one or more flexible membranes of the plurality of flexible membranes to form the barrier against the flow of fluid in one or more valve passageways of the plurality of valve passageways.

In some non-limiting embodiments or aspects, in the plurality of positions, (i) the linear actuator does not apply the force to the second face of a single flexible membrane of the plurality of flexible membranes to allow the flow of fluid in a single fluid passageway of the plurality of fluid passageways and (ii) the linear actuator applies the force to the second face of the other flexible membranes of the plurality of flexible membranes to form the barrier against the flow of fluid in the other valve passageways of the plurality of valve passageways.

In some non-limiting embodiments or aspects, the communication device includes a wireless communication device configured to wirelessly receive the control data from the external device.

In some non-limiting embodiments or aspects, the valve further includes: a housing connected to the valve body, wherein the housing includes the linear actuator, the communication device, and the processor.

In some non-limiting embodiments or aspects, the housing further includes a memory, a power source, and a user feedback device.

In some non-limiting embodiments or aspects, the valve further includes: a plurality of check valves integrated with the plurality of output ports.

In some non-limiting embodiments or aspects, the plurality of input ports is removably connected to the plurality of fluid outputs of the fluid reservoir.

In some non-limiting embodiments or aspects, the plurality of input ports is integrated with the plurality of fluid outputs of the fluid reservoir.

In some non-limiting embodiments or aspects, the fluid reservoir includes a syringe, and wherein the plurality of input ports is integrated with a plurality of tips of the syringe.

According to some non-limiting embodiments or aspects, provided is a system for maintaining multi-lumen patency, including: an actuator actuated valve, including: a valve body including a plurality of valve passageways extending between a plurality of input ports and a plurality of output ports; and a plurality of flexible membranes defining a portion of the plurality of valve passageways, wherein each flexible membrane of the plurality of flexible membranes (i) includes a first face that faces a valve passageway of the plurality of valve passageways and a second face opposite the first face and (ii) is configured for movement with respect to the valve passageway in response to a force applied to the second face of that flexible membrane to form a barrier against a flow of fluid in the valve passageway; a linear actuator configured to apply the force to the second face of each flexible membrane to form the barrier against the flow of fluid in the valve passageway; a multi-lumen catheter including a plurality of lumens is connected to the plurality of output ports; and a fluid reservoir including a plurality of fluid outputs connected to the plurality of input ports.

In some non-limiting embodiments or aspects, the fluid reservoir includes one of an IV bag, an infusion pump, and a syringe.

In some non-limiting embodiments or aspects, the system further includes: a communication device configured to receive control data from an external computing device; and a processor configured to control, based on the control data, the linear actuator between a plurality of positions in which the force is applied to one or more flexible membranes of the plurality of flexible membranes to form the barrier against the flow of fluid in one or more valve passageways of the plurality of valve passageways.

In some non-limiting embodiments or aspects, in the plurality of positions, (i) the linear actuator does not apply the force to the second face of a single flexible membrane of the plurality of flexible membranes to allow the flow of fluid in a single fluid passageway of the plurality of fluid passageways and (ii) the linear actuator applies the force to the second face of the other flexible membranes of the plurality of flexible membranes to form the barrier against the flow of fluid in the other valve passageways of the plurality of valve passageways.

In some non-limiting embodiments or aspects, the system further includes: an infusion pump including the linear actuator.

According to some non-limiting embodiments or aspects, provided is an actuator actuated valve for maintaining multi-lumen patency, including: a plurality of flexible tubes including a plurality of valve passageways extending between a plurality of input ports and a plurality of output ports, wherein the plurality of input ports are configured to be connected to a plurality of fluid outputs of a fluid reservoir; a plate including a plurality of guide walls spaced apart from each other and extending from the plate, wherein the plurality of guide walls define a plurality of guide tracks between pairs of the plurality of guide walls, and wherein the plurality of flexible tubes is received in the plurality of guide tracks between the pairs of the plurality of guide walls; and a cam shaft including a plurality of cams, wherein the cam shaft is rotatable relative to the plate between a plurality of positions in which one or more cams of the plurality of cams pinches one or more flexible tubes of the plurality of flexible tubes against the plate to form a barrier against a flow of fluid in one or more fluid passageways of the plurality of fluid passageways.

In some non-limiting embodiments or aspects, in the plurality of positions, (i) a single cam of the plurality of cams does not pinch a single flexible tube of the plurality of flexible tubes against the plate to allow the flow of fluid in a single fluid passageway of the plurality of fluid passageways and (ii) the other cams of the plurality of cams press the other flexible tubes of the plurality of flexible tubes against the plate to form barriers against the flow of fluid in the other fluid passageways of the plurality of fluid passageways.

In some non-limiting embodiments or aspects, the valve further includes: a rotary actuator configured to rotate the cam shaft relative to the plate between the plurality of positions.

In some non-limiting embodiments or aspects, the valve further includes: a communication device configured to receive control data from an external computing device; and a processor configured to control, based on the control data, the rotatory actuator to rotate the cam shaft relative to the plate between the plurality of positions.

In some non-limiting embodiments or aspects, the communication device includes a wireless communication device configured to wirelessly receive the control data from the external device.

In some non-limiting embodiments or aspects, the valve further includes: a housing connected to the valve body, wherein the housing includes the rotary actuator, the communication device, and the processor.

In some non-limiting embodiments or aspects, the housing further includes a memory, a power source, and a user feedback device.

In some non-limiting embodiments or aspects, the valve further includes: a plurality of check valves integrated with the plurality of output ports.

In some non-limiting embodiments or aspects, the plurality of input ports is removably connected to the plurality of fluid outputs of the fluid reservoir.

In some non-limiting embodiments or aspects, the plurality of input ports is integrated with the plurality of fluid outputs of the fluid reservoir.

In some non-limiting embodiments or aspects, the fluid reservoir includes a syringe.

In some non-limiting embodiments or aspects, an end of the cam shaft includes an actuator interface configured to interface with a rotary actuator.

According to some non-limiting embodiments or aspects, provided is a system, including: an actuator actuated valve, including: a plurality of flexible tubes including a plurality of valve passageways extending between a plurality of input ports and a plurality of output ports; a plate including a plurality of guide walls spaced apart from each other and extending from the plate, wherein the plurality of guide walls define a plurality of guide tracks between pairs of the plurality of guide walls, and wherein the plurality of flexible tubes is received in the plurality of guide tracks between the pairs of the plurality of guide walls; and a cam shaft including a plurality of cams, wherein the cam shaft is rotatable relative to the plate between a plurality of positions in which one or more cams of the plurality of cams pinches one or more flexible tubes of the plurality of flexible tubes against the plate to form a barrier against a flow of fluid in one or more fluid passageways of the plurality of fluid passageways; a rotary actuator configured to rotate the cam shaft relative to the plate between the plurality of positions; a fluid reservoir including a plurality of fluid outputs, wherein the plurality of input ports is connected to the plurality of fluid outputs of the fluid reservoir.

In some non-limiting embodiments or aspects, in the plurality of positions, (i) a single cam of the plurality of cams does not pinch a single flexible tube of the plurality of flexible tubes against the plate to allow the flow of fluid in a single fluid passageway of the plurality of fluid passageways and (ii) the other cams of the plurality of cams press the other flexible tubes of the plurality of flexible tubes against the plate to form barriers against the flow of fluid in the other fluid passageways of the plurality of fluid passageways.

In some non-limiting embodiments or aspects, the fluid reservoir includes one of an IV bag, an infusion pump, and a syringe.

In some non-limiting embodiments or aspects, the system further includes: a communication device configured to receive control data from an external computing device; and a processor configured to control, based on the control data, the rotary actuator to rotate the cam shaft relative to the plate between the plurality of positions.

In some non-limiting embodiments or aspects, the system further includes: an infusion pump including the rotary actuator, wherein a sidewall of the infusion pump includes the plate including the plurality of guide walls defining the plurality of guide tracks.

According to some non-limiting embodiments or aspects, provided is an infusion pump, comprising: a fluid reservoir including a plurality of fluid outputs; a plurality of flexible tubes including a plurality of valve passageways extending between a plurality of input ports and a plurality of output ports, wherein the plurality of input ports are connected to the plurality of fluid outputs of the fluid reservoir; a housing including a plate including a plurality of guide walls spaced apart from each other and extending from the plate, wherein the plurality of guide walls define a plurality of guide tracks between pairs of the plurality of guide walls, and wherein the plurality of flexible tubes is received in the plurality of guide tracks between the pairs of the plurality of guide walls; an actuator configured to pinch one or more flexible tubes of the plurality of flexible tubes at least one of against the plate and against the guide walls to form a barrier against a flow of fluid in one or more fluid passageways of the plurality of fluid passageways.

Further embodiments or aspects are set forth in the following numbered clauses:

Clause 1. An actuator actuated valve for maintaining multi-lumen patency, comprising: a valve manifold including an input port, a first output port, and a second output port; and a valve spool extending within the valve manifold, wherein the valve spool defines a first valve passageway and a second valve passageway, wherein the valve spool is rotatable relative to the valve manifold between a first position in which the input port and the first output port are in fluid communication via the first valve passageway and a second position in which the input port and the second output port are in fluid communication via the second valve passageway.

Clause 2. The actuator actuated valve of clause 1, wherein, when the valve spool is between the first position and the second position, the input port is not in fluid communication with the first output port and the second output port.

Clause 3. The actuator actuated valve of any of clauses 1 and 2, wherein, when the valve spool is between the first position and the second position, the input port is in fluid communication with each of the first output port and the second output port.

Clause 4. The actuator actuated valve of any of clauses 1-3, wherein the valve spool is rotatable relative to the valve manifold between the first position, the second position, and a third position in which the input port is not in fluid communication with the first output port and the second output port.

Clause 5. The actuator actuated valve of any of clauses 1-4, wherein, when the valve spool is in the first position, the input port is not in fluid communication with the second output port, and wherein, when the valve spool is in the second position, the input port is not in fluid communication with the first output port.

Clause 6. The actuator actuated valve of any of clauses 1-5, wherein the valve manifold further includes at least one further output port, wherein the valve spool further defines at least one further valve passageway, wherein the valve spool is further rotatable relative to the valve manifold between the first position, the second position, and at least one further position in which in the input port and the at least one further output port are in fluid communication via the at least one further valve passageway.

Clause 7. The actuator actuated valve of any of clauses 1-6, wherein the valve spool is rotatable between a plurality of positions including the first position, the second position, and the at least one further position, and wherein, in the plurality of positions, (i) a single output port of the first output port, the second output port, and the at least one further output port is in fluid communication with the input port via a single valve passageway of the first valve passageway, the second valve passageway, and the at least one further valve passageway and (ii) the other output ports of the first output port, the second output port, and the at least one further output port are not in fluid communication with the input port via the other valve passageways of the first valve passageway, the second valve passageway, and the at least one further valve passageway.

Clause 8. The actuator actuated valve of any of clauses 1-7, further comprising: a rotary actuator configured to rotate the valve spool relative to the valve manifold between the first position and the second position.

Clause 9. The actuator actuated valve of any of clauses 1-8, further comprising: a communication device configured to receive control data from an external computing device; and a processor configured to control, based on the control data, the rotary actuator to rotate the valve spool relative to the valve manifold between the first position and the second position.

Clause 10. The actuator actuated valve of any of clauses 1-9, wherein the communication device includes a wireless communication device configured to wirelessly receive the control data from the external device.

Clause 11. The actuator actuated valve of any of clauses 1-10, further comprising: a housing connected to the valve spool, wherein the housing includes the rotary actuator, the communication device, and the processor.

Clause 12. The actuator actuated valve of any of clauses 1-11, wherein the housing further includes a memory, a power source, and a user feedback device.

Clause 13. The actuator actuated valve of any of clauses 1-12, further comprising: a first check valve integrated with the first output port; a second check valve integrated with the second output port.

Clause 14. The actuator actuated valve of any of clauses 1-13, wherein the input port is removably connected to a fluid output of a fluid reservoir.

Clause 15. The actuator actuated valve of any of clauses 1-14, wherein the input port is integrated with a fluid output of a fluid reservoir.

Clause 16. The actuator actuated valve of any of clauses 1-15, further comprising: wherein the fluid reservoir includes a syringe, and wherein the input port is integrated with a tip of the syringe.

Clause 17. The actuator actuated valve of any of clauses 1-16, wherein the valve spool includes an actuator interface extending from an end of the valve spool, and wherein the actuator interface is configured to interface with a rotary actuator.

Clause 18. A system for maintaining multi-lumen patency, comprising: an actuator actuated diverter valve including: a valve manifold including an input port, a first output port, and a second output port; and a valve spool extending within the valve manifold, wherein the valve spool defines a first valve passageway and a second valve passageway, wherein the valve spool is rotatable relative to the valve manifold between a first position in which the input port and the first output port are in fluid communication via the first valve passageway and a second position in which the input port and the second output port are in fluid communication via the second valve passageway; and a rotary actuator configured to rotate the valve spool relative to the valve manifold between the first position and the second position; a multi-lumen catheter including: a first lumen connected to the first output port; and a second lumen connected to the second output port; and a fluid reservoir connected to the input port.

Clause 19. The system of clause 18, wherein the fluid reservoir includes one of an IV bag, an infusion pump, and a syringe.

Clause 20. The system of any of clauses 18 and 19, further comprising: a communication device configured to receive control data from an external computing device; and a processor configured to control, based on the control data, the rotary actuator to rotate the valve spool relative to the valve manifold between the first position and the second position.

Clause 21. The system of any of clauses 18-20, wherein the valve manifold further includes at least one further output port, wherein the valve spool further defines at least one further valve passageway, wherein the valve spool is further rotatable relative to the valve manifold between the first position, the second position, and at least one further position in which in the input port and the at least one further output port are in fluid communication via the at least one further valve passageway.

Clause 22. The system of any of clauses 18-21, wherein the valve spool is rotatable between a plurality of positions including the first position, the second position, and the at least one further position, and wherein, in the plurality of positions, (i) a single output port of the first output port, the second output port, and the at least one further output port is in fluid communication with the input port via a single valve passageway of the first valve passageway, the second valve passageway, and the at least one further valve passageway and (ii) the other output ports of the first output port, the second output port, and the at least one further output port are not in fluid communication with the input port via the other valve passageways of the first valve passageway, the second valve passageway, and the at least one further valve passageway.

Clause 23. The system of any of clauses 18-22, further comprising: an infusion pump including the rotary actuator, wherein the valve spool includes an actuator interface extending from an end of the valve spool that interfaces with the rotary actuator.

Clause 24. A method for pulsatile flow IV line flushing, comprising: (i) measuring, with an infusion pump, a baseline fluid pressure of an IV catheter line connected to the infusion pump and a patient, wherein the baseline fluid pressure is measured when a flow rate of a fluid through the IV catheter line is zero; (ii) increasing, with the infusion pump, the flow rate of the fluid through the IV catheter line until a current pressure of the IV catheter line satisfies a predetermined delta pressure setting above the measured baseline fluid pressure; (iii) in response to the current pressure of the IV catheter line satisfying the predetermined delta pressure setting above the measured baseline fluid pressure, controlling, with the infusion pump, based on the current pressure of the IV catheter line, the flow rate of the fluid through the IV catheter line to maintain the current pressure of the IV catheter line at the predetermined delta pressure setting above the baseline fluid pressure until a predetermined pulsatile flow volume is infused through the IV catheter line; (iv) in response to the predetermined pulsatile flow volume being infused into the IV catheter line, decreasing, with the infusion pump, the flow rate of the fluid through the IV catheter line until the flow rate is zero; (v) in response to decreasing the flow rate of the fluid through the IV catheter line to zero, maintaining, with the infusion pump, the flow rate of the fluid through the IV catheter line at zero for a predetermined period of time; and (vi) after the predetermined period of time, repeating steps (ii)-(v) until a predetermined total flush volume is infused through the IV catheter line.

Clause 25. The method of clause 24, wherein, if the measured baseline fluid pressure is measured as greater than a threshold baseline fluid pressure, a value of the measured baseline fluid pressure is set as the threshold baseline fluid pressure for determining the predetermined delta pressure setting above the measured baseline fluid pressure.

Clause 26. The method of any of clauses 24 and 25, wherein the infusion pump linearly increases the flow rate of the fluid through the IV catheter line until the current pressure of the IV catheter line satisfies the predetermined delta pressure setting above the measured baseline fluid pressure.

Clause 27. The method of any of clauses 24-26, wherein the infusion pump controls the flow rate of the fluid through the IV catheter line to remain at or below a safety threshold flow rate.

Clause 28. The method of any of clauses 24-26, wherein the predetermined period of time is 5 seconds, and wherein the predetermined total flush volume is in a range of 5 mL to 10 ml.

Clause 29. An infusion pump, comprising: a pressure sensor configured to measure a baseline fluid pressure of an IV catheter line connected to the infusion pump and a patient, wherein the baseline fluid pressure is measured when a flow rate of a fluid through the IV catheter line is zero; a fluid pump; and at least one processor programmed and/or configured to: (i) control the fluid pump to increase the flow rate of the fluid through the IV catheter line until a current pressure of the IV catheter line satisfies a predetermined delta pressure setting above the measured baseline fluid pressure; (ii) in response to the current pressure of the IV catheter line satisfying the predetermined delta pressure setting above the measured baseline fluid pressure, control, based on the current pressure of the IV catheter line, the fluid pump to control the flow rate of the fluid through the IV catheter line to maintain the current pressure of the IV catheter line at the predetermined delta pressure setting above the baseline fluid pressure until a predetermined pulsatile flow volume is infused through the IV catheter line; (iii) in response to the predetermined pulsatile flow volume being infused into the IV catheter line, control the fluid pump to decrease the flow rate of the fluid through the IV catheter line until the flow rate is zero; (iv) in response to decreasing the flow rate of the fluid through the IV catheter line to zero, control the fluid pump to maintain the flow rate of the fluid through the IV catheter line at zero for a predetermined period of time; and (i) after the predetermined period of time, repeat steps (i)-(iv) until a predetermined total flush volume is infused through the IV catheter line.

Clause 30. The infusion pump of clause 29, wherein the flow rate of the fluid through the IV catheter line is linearly increased until the current pressure of the IV catheter line satisfies the predetermined delta pressure setting above the measured baseline fluid pressure.

Clause 31. The infusion pump of any of clauses 29 and 30, wherein the flow rate of the fluid through the IV catheter line is controlled to remain at or below a safety threshold flow rate.

Clause 32. The infusion pump of any of clauses 29-31, wherein the predetermined period of time is 5 seconds, and wherein the predetermined total flush volume is in a range of 5 mL to 10 ml.

Clause 33. An actuator actuated valve for maintaining multi-lumen patency, comprising: a valve body including a plurality of valve passageways extending between a plurality of input ports and a plurality of output ports, wherein the plurality of input ports is configured to be connected to a plurality of fluid outputs of a fluid reservoir; and a plurality of flexible membranes defining a portion of the plurality of valve passageways, wherein each flexible membrane of the plurality of flexible membranes (i) includes a first face that faces a valve passageway of the plurality of valve passageways and a second face opposite the first face and (ii) is configured for movement with respect to the valve passageway in response to a force applied to the second face of that flexible membrane to form a barrier against a flow of fluid in the valve passageway.

Clause 34. The actuator actuated valve of clause 33, further comprising: a linear actuator configured to apply the force to the second face of each flexible membrane to form the barrier against the flow of fluid in the valve passageway.

Clause 35. The actuator actuated valve of any of clauses 33 and 34, further comprising: a communication device configured to receive control data from an external computing device; and a processor configured to control, based on the control data, the linear actuator between a plurality of positions in which the force is applied to one or more flexible membranes of the plurality of flexible membranes to form the barrier against the flow of fluid in one or more valve passageways of the plurality of valve passageways.

Clause 36. The actuator actuated valve of any of clauses 33-35, wherein, in the plurality of positions, (i) the linear actuator does not apply the force to the second face of a single flexible membrane of the plurality of flexible membranes to allow the flow of fluid in a single fluid passageway of the plurality of fluid passageways and (ii) the linear actuator applies the force to the second face of the other flexible membranes of the plurality of flexible membranes to form the barrier against the flow of fluid in the other valve passageways of the plurality of valve passageways.

Clause 37. The actuator actuated valve of any of clauses 33-36, wherein the communication device includes a wireless communication device configured to wirelessly receive the control data from the external device.

Clause 38. The actuator actuated valve of any of clauses 33-37, further comprising: a housing connected to the valve body, wherein the housing includes the linear actuator, the communication device, and the processor.

Clause 39. The actuator actuated valve of any of clauses 33-38, wherein the housing further includes a memory, a power source, and a user feedback device.

Clause 40. The actuator actuated valve of any of clauses 33-39, further comprising: a plurality of check valves integrated with the plurality of output ports.

Clause 41. The actuator actuated valve of any of clauses 33-40, wherein the plurality of input ports is removably connected to the plurality of fluid outputs of the fluid reservoir.

Clause 42. The actuator actuated valve of any of clauses 33-41, wherein the plurality of input ports is integrated with the plurality of fluid outputs of the fluid reservoir.

Clause 43. The actuator actuated valve of any of clauses 33-42, further comprising: wherein the fluid reservoir includes a syringe, and wherein the plurality of input ports is integrated with a plurality of tips of the syringe.

Clause 44. A system for maintaining multi-lumen patency, comprising: an actuator actuated valve, including: a valve body including a plurality of valve passageways extending between a plurality of input ports and a plurality of output ports; and a plurality of flexible membranes defining a portion of the plurality of valve passageways, wherein each flexible membrane of the plurality of flexible membranes (i) includes a first face that faces a valve passageway of the plurality of valve passageways and a second face opposite the first face and (ii) is configured for movement with respect to the valve passageway in response to a force applied to the second face of that flexible membrane to form a barrier against a flow of fluid in the valve passageway; a linear actuator configured to apply the force to the second face of each flexible membrane to form the barrier against the flow of fluid in the valve passageway; a multi-lumen catheter including a plurality of lumens is connected to the plurality of output ports; and a fluid reservoir including a plurality of fluid outputs connected to the plurality of input ports.

Clause 45. The system of clause 44, wherein the fluid reservoir includes one of an IV bag, an infusion pump, and a syringe.

Clause 46. The system of any of clauses 44 and 45, further comprising: a communication device configured to receive control data from an external computing device; and a processor configured to control, based on the control data, the linear actuator between a plurality of positions in which the force is applied to one or more flexible membranes of the plurality of flexible membranes to form the barrier against the flow of fluid in one or more valve passageways of the plurality of valve passageways.

Clause 47. The system of any of clauses 44-46, wherein, in the plurality of positions, (i) the linear actuator does not apply the force to the second face of a single flexible membrane of the plurality of flexible membranes to allow the flow of fluid in a single fluid passageway of the plurality of fluid passageways and (ii) the linear actuator applies the force to the second face of the other flexible membranes of the plurality of flexible membranes to form the barrier against the flow of fluid in the other valve passageways of the plurality of valve passageways.

Clause 48. The system of any of clauses 44-47, further comprising: an infusion pump including the linear actuator.

Clause 49. An actuator actuated valve for maintaining multi-lumen patency, comprising: a plurality of flexible tubes including a plurality of valve passageways extending between a plurality of input ports and a plurality of output ports, wherein the plurality of input ports are configured to be connected to a plurality of fluid outputs of a fluid reservoir; a plate including a plurality of guide walls spaced apart from each other and extending from the plate, wherein the plurality of guide walls define a plurality of guide tracks between pairs of the plurality of guide walls, and wherein the plurality of flexible tubes is received in the plurality of guide tracks between the pairs of the plurality of guide walls; and a cam shaft including a plurality of cams, wherein the cam shaft is rotatable relative to the plate between a plurality of positions in which one or more cams of the plurality of cams pinches one or more flexible tubes of the plurality of flexible tubes against the plate to form a barrier against a flow of fluid in one or more fluid passageways of the plurality of fluid passageways.

Clause 50. The actuator actuated valve of clause 49, wherein, in the plurality of positions, (i) a single cam of the plurality of cams does not pinch a single flexible tube of the plurality of flexible tubes against the plate to allow the flow of fluid in a single fluid passageway of the plurality of fluid passageways and (ii) the other cams of the plurality of cams press the other flexible tubes of the plurality of flexible tubes against the plate to form barriers against the flow of fluid in the other fluid passageways of the plurality of fluid passageways.

Clause 51. The actuator actuated valve of any of clauses 49 and 50, further comprising: a rotary actuator configured to rotate the cam shaft relative to the plate between the plurality of positions.

Clause 52. The actuator actuated valve of any of clauses 49-51, further comprising: a communication device configured to receive control data from an external computing device; and a processor configured to control, based on the control data, the rotatory actuator to rotate the cam shaft relative to the plate between the plurality of positions.

Clause 53. The actuator actuated valve of any of clauses 49-52, wherein the communication device includes a wireless communication device configured to wirelessly receive the control data from the external device.

Clause 54. The actuator actuated valve of any of clauses 49-53, further comprising: a housing connected to the valve body, wherein the housing includes the rotary actuator, the communication device, and the processor.

Clause 55. The actuator actuated valve of any of clauses 49-54, wherein the housing further includes a memory, a power source, and a user feedback device.

Clause 56. The actuator actuated valve of any of clauses 49-55, further comprising: a plurality of check valves integrated with the plurality of output ports.

Clause 57. The actuator actuated valve of any of clauses 49-56, wherein the plurality of input ports is removably connected to the plurality of fluid outputs of the fluid reservoir.

Clause 58. The actuator actuated valve of any of clauses 49-57, wherein the plurality of input ports is integrated with the plurality of fluid outputs of the fluid reservoir.

Clause 59. The actuator actuated valve of any of clauses 49-58, wherein the fluid reservoir includes a syringe.

Clause 60. The actuator actuated valve of any of clauses 49-59, wherein an end of the cam shaft includes an actuator interface configured to interface with a rotary actuator.

Clause 61. A system, comprising: an actuator actuated valve, including: a plurality of flexible tubes including a plurality of valve passageways extending between a plurality of input ports and a plurality of output ports; a plate including a plurality of guide walls spaced apart from each other and extending from the plate, wherein the plurality of guide walls define a plurality of guide tracks between pairs of the plurality of guide walls, and wherein the plurality of flexible tubes is received in the plurality of guide tracks between the pairs of the plurality of guide walls; and a cam shaft including a plurality of cams, wherein the cam shaft is rotatable relative to the plate between a plurality of positions in which one or more cams of the plurality of cams pinches one or more flexible tubes of the plurality of flexible tubes against the plate to form a barrier against a flow of fluid in one or more fluid passageways of the plurality of fluid passageways; a rotary actuator configured to rotate the cam shaft relative to the plate between the plurality of positions; a fluid reservoir including a plurality of fluid outputs, wherein the plurality of input ports is connected to the plurality of fluid outputs of the fluid reservoir.

Clause 62. The system of clause 61, wherein, in the plurality of positions, (i) a single cam of the plurality of cams does not pinch a single flexible tube of the plurality of flexible tubes against the plate to allow the flow of fluid in a single fluid passageway of the plurality of fluid passageways and (ii) the other cams of the plurality of cams press the other flexible tubes of the plurality of flexible tubes against the plate to form barriers against the flow of fluid in the other fluid passageways of the plurality of fluid passageways.

Clause 63. The system of any of clauses 61 and 62, wherein the fluid reservoir includes one of an IV bag, an infusion pump, and a syringe.

Clause 64. The system of any of clauses 61-63, further comprising: a communication device configured to receive control data from an external computing device; and a processor configured to control, based on the control data, the rotary actuator to rotate the cam shaft relative to the plate between the plurality of positions.

Clause 65. The system of any of clauses 61-64, further comprising: an infusion pump including the rotary actuator, wherein a sidewall of the infusion pump includes the plate including the plurality of guide walls defining the plurality of guide tracks.

Clause 66. An infusion pump, comprising: a fluid reservoir including a plurality of fluid outputs; a plurality of flexible tubes including a plurality of valve passageways extending between a plurality of input ports and a plurality of output ports, wherein the plurality of input ports are connected to the plurality of fluid outputs of the fluid reservoir; a housing including a plate including a plurality of guide walls spaced apart from each other and extending from the plate, wherein the plurality of guide walls define a plurality of guide tracks between pairs of the plurality of guide walls, and wherein the plurality of flexible tubes is received in the plurality of guide tracks between the pairs of the plurality of guide walls; an actuator configured to pinch one or more flexible tubes of the plurality of flexible tubes at least one of against the plate and against the guide walls to form a barrier against a flow of fluid in one or more fluid passageways of the plurality of fluid passageways.

These and other features and characteristics of the present disclosure, as well as the methods of operation and functions of the related elements of structures and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of limits. As used in the specification and the claims, the singular form of “a,” “an,” and “the” include plural referents unless the context clearly dictates otherwise.

### BRIEF DESCRIPTION OF THE DRAWINGS

Additional advantages and details of embodiments or aspects of the present disclosure are explained in greater detail below with reference to the exemplary embodiments that are illustrated in the accompanying schematic figures, in which:

FIG. 1A is a perspective view of non-limiting embodiments or aspects of an actuator actuated valve for maintaining multi-lumen patency;

FIGS. 1B and 1C are sectional views of non-limiting embodiments or aspects of an actuator actuated valve for maintaining multi-lumen patency illustrating two different positions for a valve spool rotatable relative to a valve manifold;

FIG. 1D is a diagram of non-limiting embodiments or aspects of components of a housing of an actuator actuated valve for maintaining multi-lumen patency;

FIGS. 1E and 1F are sectional views of non-limiting embodiments or aspects of an actuator actuated valve for maintaining multi-lumen patency;

FIG. 1G is a perspective view of non-limiting embodiments or aspects of a valve spool of an actuator actuated valve for maintaining multi-lumen patency;

FIG. 2 is a diagram of non-limiting embodiments or aspects of an environment in which systems, devices, products, apparatus, and/or methods, described herein, may be implemented;

FIG. 3 is a diagram of non-limiting embodiments or aspects of components of one or more devices and/or one or more systems of FIGS. 1A-1G and 2;

FIG. 4 is a flowchart of non-limiting embodiments or aspects of a process for pulsatile flow lumen flushing;

FIG. 5 is a graph illustrating steps of stages of non-limiting embodiments or aspects of a process for pulsatile lumen line flushing;

FIGS. 6A and 6B are sectional views of non-limiting embodiments or aspects of an actuator actuated valve for maintaining multi-lumen patency;

FIGS. 7A and 7B are sectional views of non-limiting embodiments or aspects of an actuator actuated valve for maintaining multi-lumen patency; and

FIG. 7C is a perspective view of non-limiting embodiments or aspects of an infusion pump including an actuator actuated valve for maintaining multi-lumen patency.

### DETAILED DESCRIPTION

It is to be understood that the present disclosure may assume various alternative variations and step sequences, except where expressly specified to the contrary. It is also to be understood that the specific devices and processes illustrated in the attached drawings, and described in the following specification, are simply exemplary and non-limiting embodiments or aspects. Hence, specific dimensions and other physical characteristics related to the embodiments or aspects disclosed herein are not to be considered as limiting.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary aspects of the invention. Hence, specific dimensions and other physical characteristics related to the aspects disclosed herein are not to be considered as limiting. All numbers and ranges used in the specification and claims are to be understood as being modified in all instances by the term “about”. By “about” is meant plus or minus twenty-five percent of the stated value, such as plus or minus ten percent of the stated value. However, this should not be considered as limiting to any analysis of the values under the doctrine of equivalents.

Unless otherwise indicated, all ranges or ratios disclosed herein are to be understood to encompass the beginning and ending values and any and all subranges or sub-ratios subsumed therein. For example, a stated range or ratio of “1 to 10” should be considered to include any and all subranges or sub-ratios between (and inclusive of) the minimum value of 1 and the maximum value of 10; that is, all subranges or sub-ratios beginning with a minimum value of 1 or more and ending with a maximum value of 10 or less. The ranges and/or ratios disclosed herein represent the average values over the specified range and/or ratio.

The terms “first”, “second”, and the like are not intended to refer to any particular order or chronology, but refer to different conditions, properties, or elements.

No aspect, component, element, structure, act, step, function, instruction, and/or the like used herein should be construed as critical or essential unless explicitly described as such. Also, as used herein, the articles “a” and “an” are intended to include one or more items, and may be used interchangeably with “one or more” and “at least one.” Furthermore, as used herein, the term “set” is intended to include one or more items (e.g., related items, unrelated items, a combination of related and unrelated items, etc.) and may be used interchangeably with “one or more” or “at least one.” Where only one item is intended, the term “one” or similar language is used. Also, as used herein, the terms “has,” “have,” “having,” or the like are intended to be open-ended terms. Further, the phrase “based on” is intended to mean “based at least in partially on” unless explicitly stated otherwise.

As used herein, the terms “communication” and “communicate” refer to the receipt or transfer of one or more signals, messages, commands, or other type of data. For one unit (e.g., any device, system, or component thereof) to be in communication with another unit means that the one unit is able to directly or indirectly receive data from and/or transmit data to the other unit. This may refer to a direct or indirect connection that is wired and/or wireless in nature. Additionally, two units may be in communication with each other even though the data transmitted may be modified, processed, relayed, and/or routed between the first and second unit. For example, a first unit may be in communication with a second unit even though the first unit passively receives data and does not actively transmit data to the second unit. As another example, a first unit may be in communication with a second unit if an intermediary unit processes data from one unit and transmits processed data to the second unit. It will be appreciated that numerous other arrangements are possible.

It will be apparent that systems and/or methods, described herein, can be implemented in different forms of hardware, software, or a combination of hardware and software. The actual specialized control hardware or software code used to implement these systems and/or methods is not limiting of the implementations. Thus, the operation and behavior of the systems and/or methods are described herein without reference to specific software code, it being understood that software and hardware can be designed to implement the systems and/or methods based on the description herein.

Some non-limiting embodiments or aspects are described herein in connection with thresholds. As used herein, satisfying a threshold may refer to a value being greater than the threshold, more than the threshold, higher than the threshold, greater than or equal to the threshold, less than the threshold, fewer than the threshold, lower than the threshold, less than or equal to the threshold, equal to the threshold, etc.

As used herein, the term “computing device” or “computer device” may refer to one or more electronic devices that are configured to directly or indirectly communicate with or over one or more networks. The computing device may be a mobile device, a desktop computer, or the like. Furthermore, the term “computer” may refer to any computing device that includes the necessary components to receive, process, and output data, and normally includes a display, a processor, a memory, an input device, and a network interface. An “application” or “application program interface” (API) refers to computer code or other data sorted on a computer-readable medium that may be executed by a processor to facilitate the interaction between software components, such as a client-side front-end and/or server-side back-end for receiving data from the client. An “interface” refers to a generated display, such as one or more graphical user interfaces (GUIs) with which a user may interact, either directly or indirectly (e.g., through a keyboard, mouse, touchscreen, etc.).

Non-limiting embodiments or aspects of the present disclosure may provide a rotary actuator (e.g., a servo, etc.) and/or a linear actuator actuated valve for maintaining multi-lumen IV catheter patency that improves multi-lumen IV catheter patency during Keep Vein Open (KVO) saline infusion and/or single medication and/or lumen infusion applications. Non-limiting embodiments or aspects of the present disclosure may enable diverting saline and/or medication flow from a single input (e.g., an IV Bag, an IV pump, a syringe, etc.) to two or more IV Catheter lumens by using a servo actuated fluid diverter manifold configured to switch incoming fluid flow to two or more output ports, which enables a clinician to ensure that each catheter lumen is receiving flow, which may be necessary to maintain patency, without additional flushing or IV maintenance. Non-limiting embodiments or aspects of the present disclosure may enable diverting saline and/or medication flow from a plurality of inputs to a single output of a plurality of outputs, which likewise enables a clinician to individually ensure that each catheter lumen is receiving flow, while avoiding a scenario in which a fluid flow is not equally distributed between multiple flow channels due to a higher flow being directed to a lumen with the least restriction.

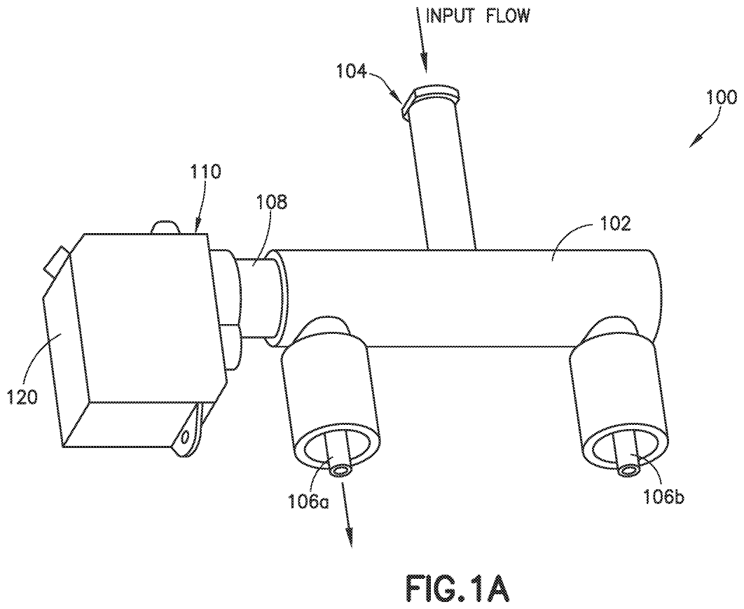

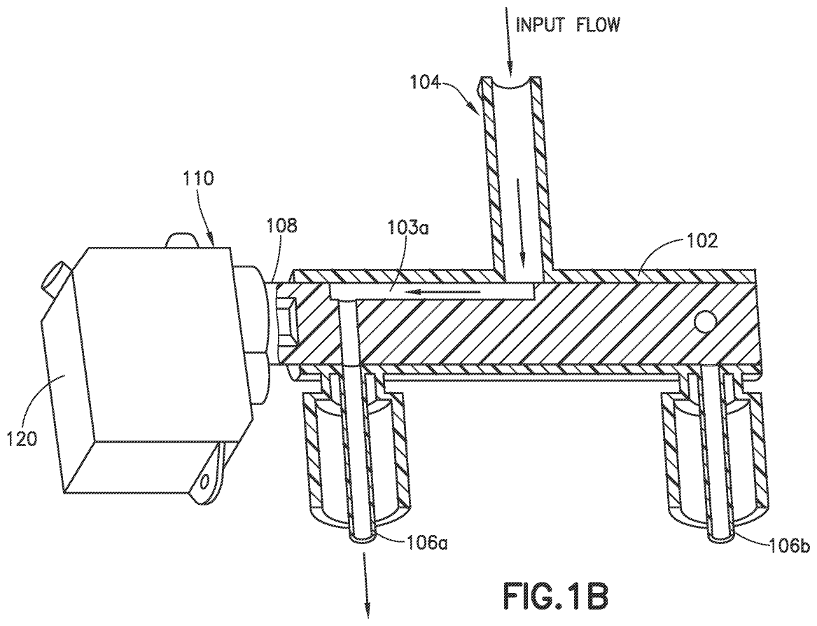

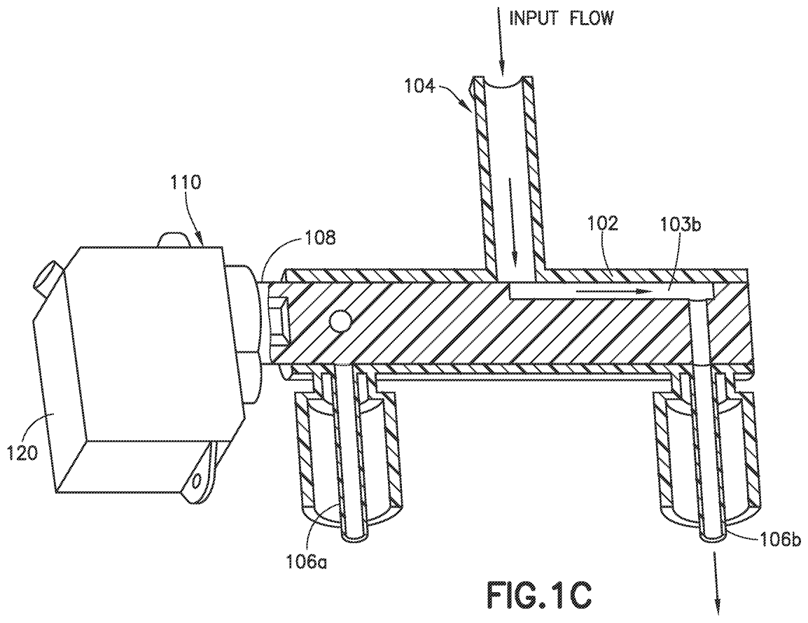

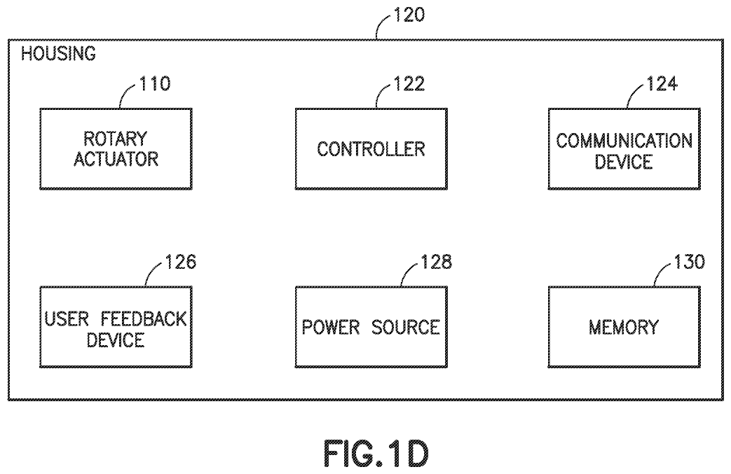

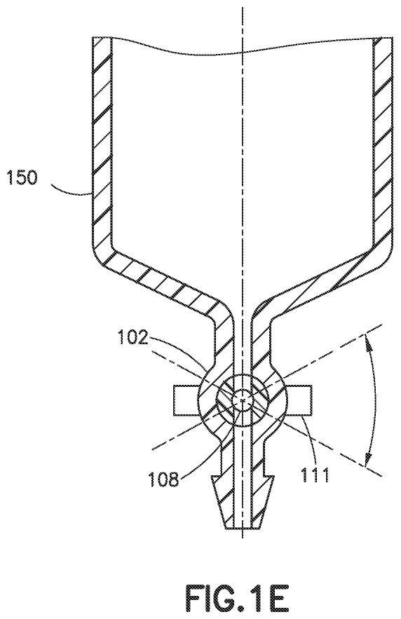

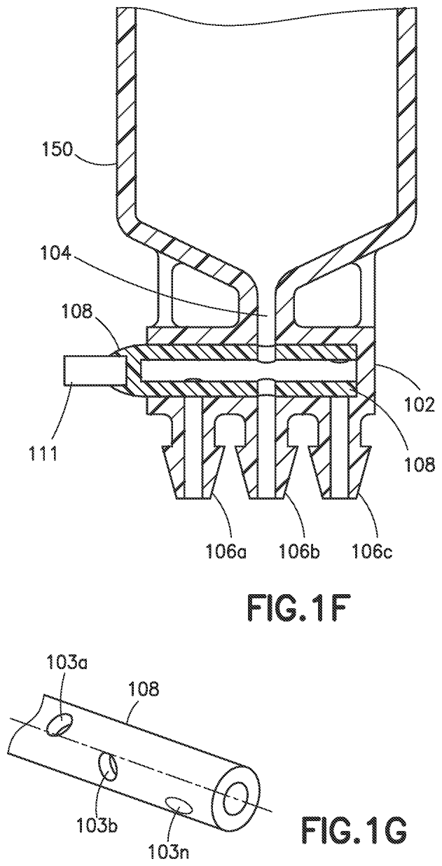

Referring to FIGS. 1A-1G, actuator actuated valve 100 (e.g., a rotary actuator actuated diverter valve, etc.) for maintaining multi-lumen patency may include valve manifold 102, valve spool 108, actuator 110 (e.g., a rotary actuator, etc.), and/or housing 120.

Valve manifold 102 may include an input port 104, a first output port 106a, and/or a second output port 106b.

Valve spool 108 may extend within the valve manifold. Valve spool 108 may define first valve passageway 103a and second valve passageway 103b. Valve spool 108 may be rotatable relative to valve manifold 102 between a first position in which input port 104 and first output port 106a are in fluid communication via first valve passageway 103a (e.g., as shown in FIG. 1B) and a second position in which the input port 104 and second output port 106b are in fluid communication via second valve passageway 103b (e.g., as shown in FIG. 1C).

Rotary actuator 110 may be configured to rotate valve spool 108 relative to valve manifold 102 between the first position and the second position. For example, rotary actuator 110 may include a servomotor. As an example, an end of valve spool 108 may include an actuator interface 111 configured to interface with rotatory actuator 110 (e.g., a complementary mechanical mating, etc.).

In some non-limiting embodiments or aspects, when valve spool 108 is between the first position and the second position, input port 104 is not in fluid communication with first output port 106a and second output port 106b. For example, when valve spool 108 is transitioning in-between positions, valve spool 108 may block fluid flow (e.g., break before make, etc.) from first output port 106a and second output port 106b.

In some non-limiting embodiments or aspects, when valve spool 108 is between the first position and the second position, input port 104 is in fluid communication with each of first output port 106a and second output port 106b. For example, when valve spool 108 is transitioning in-between positions, valve spool 108 may allow fluid flow (e.g., make before break, etc.) from first output port 106a and second output port 106b.

In some non-limiting embodiments or aspects, valve spool 108 is rotatable relative to valve manifold 102 between the first position, the second position, and a third position in which input port 104 is not in fluid communication with first output port 106a and second output port 106b.

In some non-limiting embodiments or aspects, when valve spool 108 is in the first position, input port 104 is not in fluid communication with second output port 106b, and, when valve spool 108 is in the second position, input port 104 is not in fluid communication with first output port 106a.

In some non-limiting embodiments or aspects, valve manifold 102 further includes at least one further output port 106n, valve spool 108 further defines at least one further valve passageway 103n, valve spool 108 is further rotatable relative to valve manifold 102 between the first position, the second position, and at least one further position in which in input port 104 and the at least one further output port 106n are in fluid communication via the at least one further valve passageway 103n (e.g., as shown in FIGS. 1E-1G). For example, valve spool 108 may be rotatable between a plurality of positions, wherein, in the plurality of positions, (i) a single output port of the plurality of output ports 106a, 106b, 106n is in fluid communication with input port 104 via a single valve passageway and (ii) the other output ports of the plurality of output ports 106a, 106b, and 106n are not in fluid communication with the input port 104 via the other valve passageways.

In some non-limiting embodiments or aspects, a first check valve is integrated with and/or connected to first output port 106a, and a second check valve is integrated with second output port 106b. For example, one or more check valves may be integrated with and/or connected to one or more output ports of valve manifold 102.

In some non-limiting embodiments or aspects, input port 104 is removably connected to a fluid output of fluid reservoir 150. In some non-limiting embodiments or aspects, input port 104 is integrated with a fluid output of fluid reservoir 150 (e.g., as shown in FIGS. 1E and 1F). For example, input port 104, valve manifold 102, and/or output ports 106a, 106b, 106n may be integrated with (e.g., permanently connected to, molded as a single piece with, etc.) fluid reservoir 150 (e.g., with a tip of a syringe, etc.).

As shown in FIG. 1D, housing 120 may include actuator 110, controller 122, communication device 124, user feedback device 126, power source 128, and/or memory 130. For example, housing 120 may be attached to (e.g., removably attached to, integrated with, etc.) valve spool 108. In some non-limiting embodiments or aspects, housing 120 (and/or its included components) is a reusable housing that is configured to be attached to and used with a first valve manifold 102 and valve spool 108 combination, removed from the first valve manifold 102 and valve spool 108 combination after use, and subsequently attached to and used with a different valve manifold 102 and valve spool 108 combination. In some non-limiting embodiments or aspects, housing 12 (and/or its included components) may be a disposable housing that is configured to be disposed of with valve manifold 102 and valve spool 108 combination after a single use. In some non-limiting embodiments or aspects, housing 120 may include a housing of an infusion pump.

Controller 122 may be programmed and/or configured to control, based on control data, rotary actuator 110 to rotate valve spool 108 relative to valve manifold 102 between the first position and the second position. In some non-limiting embodiments or aspects, processor 124 may include a low power microcontroller unit (MCU). Controller 122 may store information associated with a position of valve spool 108 in memory 130.

Communication device 124 may be configured to communicate with an external computing device (e.g., infusion pump 202, etc.). For example, communication device 124 may be configured to receive control data from the external computing device, the control data including program instructions, which when executed by controller 122, cause controller 122 to control rotary actuator 110 in accordance with the program instructions. In some non-limiting embodiments or aspects, communication device 124 may include a wired communication configured to receive the control data from the external computing device via a wired connection. In some non-limiting embodiments or aspects, communication device 124 may include a wireless communication configured to wirelessly receive the control data from the external computing device and/or to communicate information associated with a position of valve spool 108 to the external computing device. In some non-limiting embodiments or aspects, communication device 124 includes one or more computing devices, chips, contactless transmitters, contactless transceivers, NFC transmitters, RFID transmitters, contact based transmitters, Bluetooth transceivers® and/or the like that enables communication device 124 to receive information directly from and/or communicate information directly to the external computing device via a short range wireless communication connection (e.g., a communication connection that uses NFC protocol, a communication connection that uses Radio-frequency identification (RFID), a communication connection that uses a Bluetooth® wireless technology standard, and/or the like).

User feedback device 126 may be configured to provide an indication associated with a position of valve spool 108 to a user. For example, user feedback device 128 may include at least one of the following: a display, a light-emitting diode (LED), an audio output device (e.g., a buzzer, a speaker, etc.), or any combination thereof.

Power source 128 may be configured to power rotary actuator 110, controller 122, communication device 124, user feedback device 126, power source 128, and/or memory 130. For example, power source 130 may include a battery (e.g., a rechargeable battery, a disposable battery, etc.).

Memory 130 may be configured to store information associated with a position of valve spool 108. In some non-limiting embodiments or aspects, memory 130 may store a current position of valve spool 108 and/or a log of positions of valve spool 108 over a period of time.

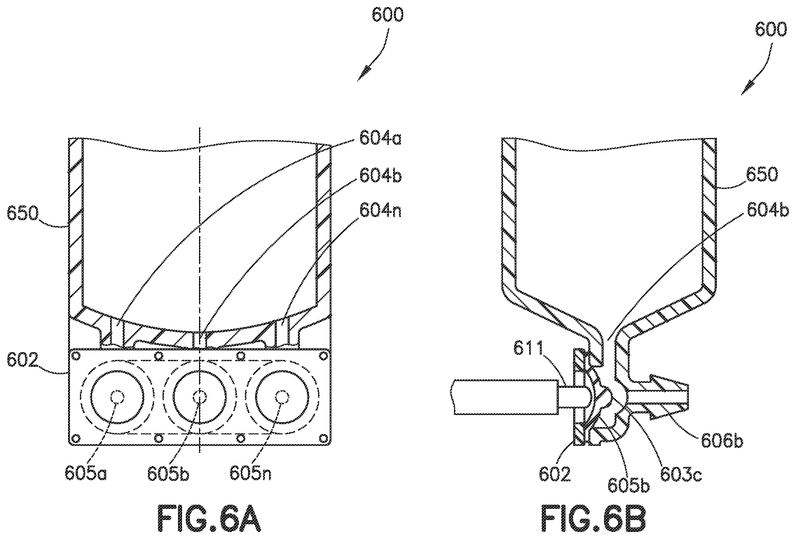

Referring now to FIGS. 6A and 6B, and still referring to FIG. 1D, an actuator actuated valve 600 for maintaining multi-lumen patency may include valve body 602, a plurality of flexible membranes 605a, 605b, 605n, actuator 610 (e.g., a linear actuator, etc.), and/or housing 620.

Valve body 602 may include a plurality of valve passageways 603a, 603b, 603n extending between a plurality of input ports 604a, 604b, 604n, a plurality of output ports 606a, 606b, 606n.

The plurality of flexible membranes 605a, 605b, 605n may define a portion of the plurality of valve passageways 603a, 603b, 603n. Each flexible membrane of the plurality of flexible membranes 605a, 605b, 605n (i) may include a first face that faces a valve passageway of the plurality of valve passageways 603a, 603b, 603n and a second face opposite the first face and (ii) may be configured for movement with respect to the valve passageway in response to a force applied to the second face of that flexible membrane to form a barrier against a flow of fluid in the valve passageway. For example, and referring specifically toe FIG. 6B, flexible membrane 605b may be configured to move inward toward an opposite wall of fluid passageway 603b and/or toward output port 604b (e.g., in a fluid passageway structure in an ‘L’ shape or right angle as shown in FIG. 6B, to contact or form a seal with the opposite wall of fluid passageway 603b and/or to plug output port 604b to form a barrier against the flow of fluid in valve passageway 603b.

Linear actuator 610 may be configured to apply a force to the second face of each flexible membrane of the plurality of flexible membranes 605a, 605b, 605n to form the barrier against the flow of fluid in the valve passageway of that flexible membrane.

As shown in FIG. 1D, housing 620 may include the same or similar component as housing 120 (e.g., controller 122, communication device 124, user feedback device 126, power source 128, memory 130, etc.) and actuator 610 (e.g., a linear actuator, etc.) instead of actuator 110. A discussion of the same or similar components and their functions is not repeated in the interest of brevity. In such an example, communication device 124 may be configured to receive control data from an external computing device, and/or controller 122 may be configured to control, based on the control data, linear actuator 610 between a plurality of positions in which the force is applied to one or more flexible membranes of the plurality of flexible membranes 605a, 605b, 605n to form the barrier against the flow of fluid in one or more valve passageways of the plurality of valve passageways 603a, 603b, 603n. In some non-limiting embodiments or aspects, housing 620 may include a housing of an infusion pump.

In some non-limiting embodiments or aspects, in the plurality of positions, (i) linear actuator 610 does not apply the force to the second face of a single flexible membrane of the plurality of flexible membranes 605a, 605b, 605n to allow the flow of fluid in a single fluid passageway of the plurality of fluid passageways 603a, 603b, 603n and/or (ii) the linear actuator applies the force to the second face of the other flexible membranes of the plurality of flexible membranes 605a, 605b, 605n to form the barrier against the flow of fluid in the other valve passageways of the plurality of valve passageways 603a, 603b, 603n. For example, if controller 122 controls linear actuator 610 to not apply the force to the second face of flexible membrane 605b to allow the flow of fluid in fluid in valve passageway 603b, controller 122 may control linear actuator 610 to apply the force to the second faces of flexible membranes 605a and 605n to form the barrier against the flow of fluid in valve passageways 603a and 603n, thereby enabling fluid flow from a plurality of inputs to a single output of a plurality of outputs while avoiding a scenario in which a fluid flow is not equally distributed between multiple flow channels due to a higher flow being directed to a lumen with the least restriction.

In some non-limiting embodiments or aspects, a plurality of check valves is connected to and/or integrated with the plurality of output ports 606a, 606b, 606n.

In some non-limiting embodiments or aspects, the plurality of input ports 604a, 604b, 604n is removably connected to a plurality of fluid outputs of fluid reservoir 650 (e.g. an IV bag, an infusion pump, a syringe, etc.). In some non-limiting embodiments or aspects, the plurality of input ports 604a, 604b, 604n is integrated with (e.g., permanently connected to, formed or molded as a single piece with, etc.) a plurality of fluid outputs of fluid reservoir 650 (e.g. an IV bag, an infusion pump, a syringe, etc.). For example, fluid reservoir 650 may include a syringe, and the plurality of input ports 604a, 604b, 604n may be removably connected to or integrated with a plurality of tips of the syringe. In some non-limiting embodiments or aspects, a multi-lumen catheter including a plurality of lumens is connected to the plurality of output ports 606a, 606b, 606n.

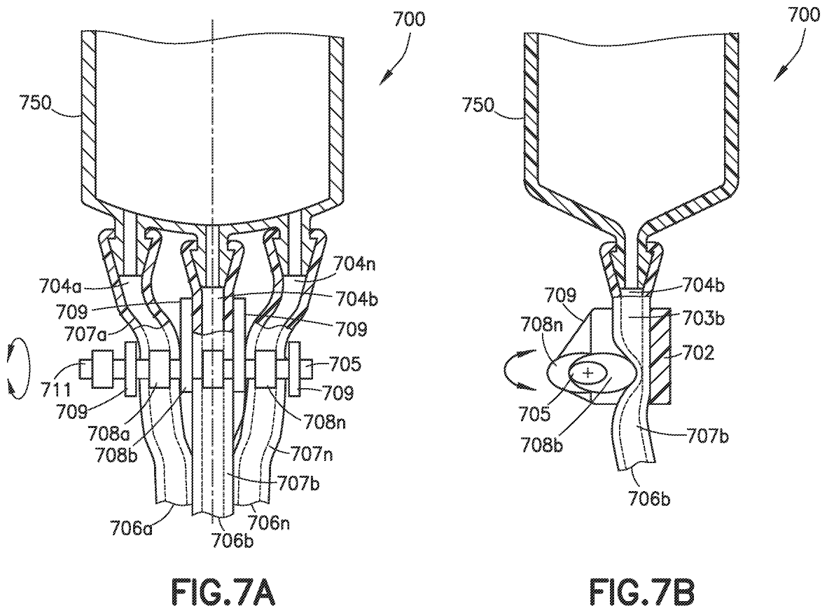

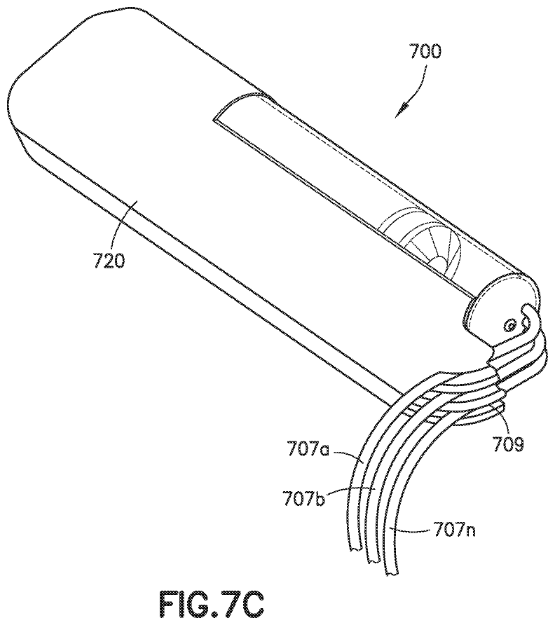

Referring now to FIGS. 7A-7C, and still referring to FIG. 1D, an actuator actuated valve 700 for maintaining multi-lumen patency may include plate 702 (e.g., a rigid plate, a base, etc.), a plurality of flexible tubes 707a, 707b, 707n, cam shaft 705, actuator 710 (e.g., a rotary actuator, etc.), and/or housing 720.

The plurality of flexible tubes 707a, 707b, 707n may include a plurality of valve passageways 703a, 703b, 703n extending between a plurality of input ports 704a, 704b, 704n and a plurality of output ports 706a, 706b, 706n. The plurality of input ports 704a, 704b, 704n may be configured to be connected to a plurality of fluid outputs of fluid reservoir 750. For example, the plurality of input ports 704a, 704b, 704n may removably connected to the plurality of fluid outputs of fluid reservoir 750. As an example, the plurality of input ports 704a, 704b, 704n may be integrated with the plurality of fluid outputs of the fluid reservoir.

Plate 702 may include a plurality of guide walls 709 spaced apart from each other and extending from plate 702. The plurality of guide walls 709 may define a plurality of guide tracks between pairs of the plurality of guide walls 709. The plurality of flexible tubes 707a, 707b, 707n may be received in the plurality of guide tracks between the pairs of the plurality of guide walls 709. For example, the plurality of flexible tubes 707a, 707b, 707n may be press fit in the plurality of guide tracks between the pairs of the plurality of guide walls 709.

Cam shaft 705 may include a plurality of cams 708a, 708b, 708n. Cam shaft 705 may be rotatable relative to plate 702 between a plurality of positions in which one or more cams of the plurality of cams 708a, 708b, 708n pinches one or more flexible tubes of the plurality of flexible tubes 707a, 707b, 707n against plate 702 to form a barrier against a flow of fluid in one or more fluid passageways of the plurality of fluid passageways 703a, 703b, 703n.

In some non-limiting embodiments or aspects, in the plurality of positions, (i) a single cam of the plurality of cams 708a, 708b, 708n does not pinch a single flexible tube of the plurality of flexible tubes 707a, 707b, 707n against plate 702 to allow the flow of fluid in a single fluid passageway of the plurality of fluid passageways 703a, 703b, 703n and (ii) the other cams of the plurality of cams 708a, 708b, 708n press the other flexible tubes of the plurality of flexible tubes 707a, 707b, 707n against plate 702 to form barriers against the flow of fluid in the other fluid passageways of the plurality of fluid passageways 703a, 703b, 703n. For example, when cam 708n does not pinch flexible tube 707n against plate 702 to allow the flow of fluid in fluid passageway 703a, cams 708a and 708b may press flexible tubes 707a and 707b against plate 702 to form barriers against the flow of fluid in fluid passageways 703a and 703b, thereby enabling fluid flow from a plurality of inputs to a single output of a plurality of outputs while avoiding a scenario in which a fluid flow is not equally distributed between multiple flow channels due to a higher flow being directed to a lumen with the least restriction.

Rotary actuator 710 may be configured to rotate cam shaft 705 relative to plate 702 between the plurality of positions. For example, an end of cam shaft 705 may include an actuator interface 711 configured to interface with a rotary actuator 710 (e.g., a complementary mechanical mating, etc.).

As shown in FIG. 1D, housing 720 may include the same or similar component as housing 120 (e.g., controller 122, communication device 124, user feedback device 126, power source 128, memory 130, etc.) and actuator 710 (e.g., a rotary actuator, etc.) instead of actuator 110. A discussion of the same or similar components and their functions is not repeated in the interest of brevity. In such an example, communication device 124 may be configured to receive control data from an external computing device, and/or controller 122 may be configured to control, based on the control data, rotatory actuator 710 to rotate cam shaft 705 relative to plate 702 between the plurality of positions. In some non-limiting embodiments or aspects, housing 720 may include a housing of an infusion pump. For example, and referring specifically to FIG. 7C, a sidewall of the housing of the infusion pump 720 may include plate 702 including the plurality of guide walls 709 defining the plurality of guide tracks. In such an example, infusion pump may include a rotary actuator mechanism (e.g., actuator 710 and cam shaft 705, etc.) and/or a linear actuator (e.g., actuator 610, etc.) configured to pinch one or more flexible tubes of the plurality of flexible tubes 707a, 707b, 707n at least one of against plate 702 and against the guide walls 709 to form a barrier against a flow of fluid in one or more fluid passageways of the plurality of fluid passageways 703a, 703b, 703n

In some non-limiting embodiments or aspects, a plurality of check valves is connected to and/or integrated with the plurality of output ports 706a, 706b, 706n.

In some non-limiting embodiments or aspects, the plurality of input ports 704a, 704b, 704n is removably connected to a plurality of fluid outputs of fluid reservoir 750 (e.g. an IV bag, an infusion pump, a syringe, etc.). In some non-limiting embodiments or aspects, the plurality of input ports 704a, 704b, 704n is integrated with (e.g., permanently connected to, formed or molded as a single piece with, etc.) a plurality of fluid outputs of fluid reservoir 750 (e.g. an IV bag, an infusion pump, a syringe, etc.). For example, fluid reservoir 750 may include a syringe, and the plurality of input ports 704a, 704b, 704n may be removably connected to or integrated with a plurality of tips of the syringe. In some non-limiting embodiments or aspects, a multi-lumen catheter including a plurality of lumens is connected to the plurality of output ports 706a, 706b, 706n.

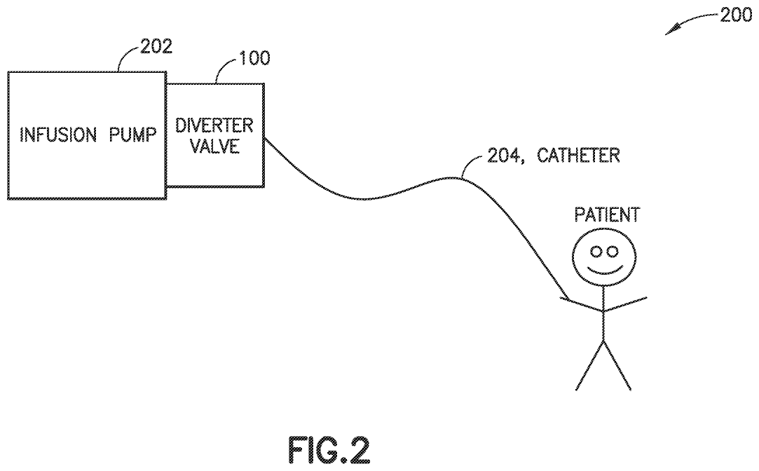

Referring also to FIG. 2, FIG. 2 is a diagram of an example environment 200 in which systems, methods, products, apparatuses, and/or devices described herein, may be implemented. As shown in FIG. 2, environment 200 may include infusion pump 202, actuator actuated valve 100, 600, and/or 700, and/or catheter 204 (e.g., a single lumen catheter, a multi-lumen catheter, etc.). For example, a system for maintaining multi-lumen patency may include infusion pump 202, actuator actuated valve 100, 600, and/or 700, and/or catheter 204. As an example, catheter 204 may include a multi-lumen catheter, and the multi-lumen catheter may include two or more lumens connected at first ends to two or more output ports 106a, 106b of valve manifold 102 (e.g., a first lumen connected to first output port 106a, a second lumen connected to the second output port 106b, etc.), to two or more output ports 606a, 606b, 606n of valve body 602, and/or two or more output ports 706a, 706b, 706n of flexible tubes 707a, 707b, 707n, and connected at seconds ends to a patient.

Infusion pump 202 may include a fluid reservoir 150, 650, and/or 750 connected to input port 104 of valve manifold 102, to input ports 604a, 604b, 604n of valve body 602, and/or to input ports 704a, 704b, 704n of flexible tubes 707a, 707b, 707. For example, a fluid reservoir 150, 650, and/or 750 may include an IV bag, an infusion pump, and/or a syringe.

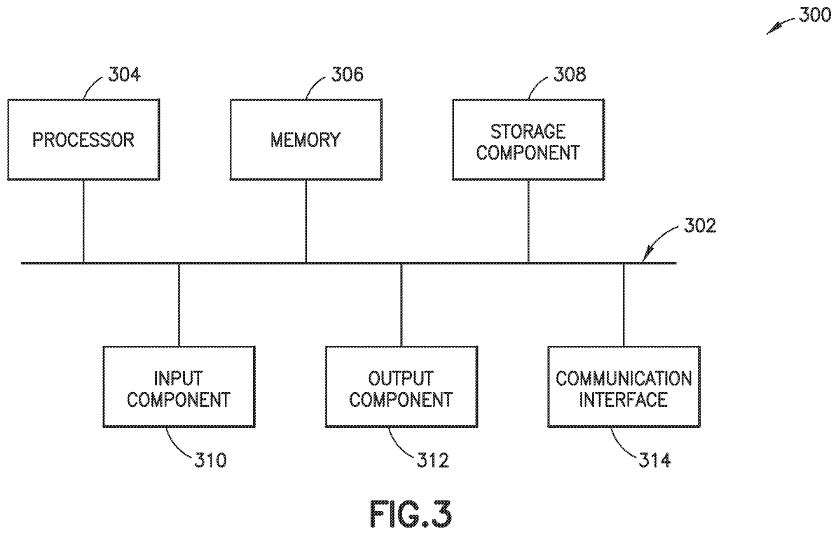

Referring now to FIG. 3, FIG. 3 is a diagram of example components of a device 300. Device 300 can correspond to one or more devices of housing 120, 620, and/or 702 and/or one or more devices of infusion pump 202. In some non-limiting embodiments or aspects, one or more devices of housing 120, 620, and/or 720 and/or one or more devices of infusion pump 202 can include at least one device 300 and/or at least one component of device 300. As shown in FIG. 3, device 300 includes bus 302, processor 304, memory 306, storage component 308, input component 310, output component 312, and communication interface 314.

Bus 302 includes a component that permits communication among the components of device 300. In some non-limiting embodiments or aspects, processor 304 is implemented in hardware, firmware, or a combination of hardware and software. For example, processor 304 includes a processor (e.g., a central processing unit (CPU), a graphics processing unit (GPU), an accelerated processing unit (APU), etc.), a microprocessor, a digital signal processor (DSP), and/or any processing component (e.g., a field-programmable gate array (FPGA), an application-specific integrated circuit (ASIC), etc.) that can be programmed to perform a function. Memory 306 includes a random access memory (RAM), a read only memory (ROM), and/or another type of dynamic or static storage device (e.g., flash memory, magnetic memory, optical memory, etc.) that stores information and/or instructions for use by processor 304.

Storage component 308 stores information and/or software related to the operation and use of device 300. For example, storage component 308 includes a hard disk (e.g., a magnetic disk, an optical disk, a magneto-optic disk, a solid state disk, etc.), a compact disc (CD), a digital versatile disc (DVD), a floppy disk, a cartridge, a magnetic tape, and/or another type of computer-readable medium, along with a corresponding drive.

Input component 310 includes a component that permits device 300 to receive information, such as via user input (e.g., a touch screen display, a keyboard, a keypad, a mouse, a button, a switch, a microphone, etc.). Additionally, or alternatively, input component 310 includes a sensor for sensing information (e.g., a global positioning system (GPS) component, an accelerometer, a gyroscope, an actuator, etc.). Output component 312 includes a component that provides output information from device 300 (e.g., a display, a speaker, one or more light-emitting diodes (LEDs), etc.).

Communication interface 314 includes a transceiver-like component (e.g., a transceiver, a separate receiver and transmitter, etc.) that enables device 300 to communicate with other devices, such as via a wired connection, a wireless connection, or a combination of wired and wireless connections. Communication interface 314 can permit device 300 to receive information from another device and/or provide information to another device. For example, communication interface 314 includes an Ethernet interface, an optical interface, a coaxial interface, an infrared interface, a radio frequency (RF) interface, a universal serial bus (USB) interface, a Wi-Fi interface, a cellular network interface, and/or the like.

Device 300 can perform one or more processes described herein. Device 300 can perform these processes based on processor 304 executing software instructions stored by a computer-readable medium, such as memory 306 and/or storage component 308. A computer-readable medium (e.g., a non-transitory computer-readable medium) is defined herein as a non-transitory memory device. A memory device includes memory space located inside of a single physical storage device or memory space spread across multiple physical storage devices.

Software instructions can be read into memory 306 and/or storage component 308 from another computer-readable medium or from another device via communication interface 314. When executed, software instructions stored in memory 306 and/or storage component 308 cause processor 304 to perform one or more processes described herein. Additionally, or alternatively, hardwired circuitry can be used in place of or in combination with software instructions to perform one or more processes described herein. Thus, embodiments described herein are not limited to any specific combination of hardware circuitry and software.

The number and arrangement of components shown in FIG. 3 are provided as an example. In some non-limiting embodiments or aspects, device 300 includes additional components, fewer components, different components, or differently arranged components than those shown in FIG. 3. Additionally, or alternatively, a set of components (e.g., one or more components) of device 300 can perform one or more functions described as being performed by another set of components of device 300.

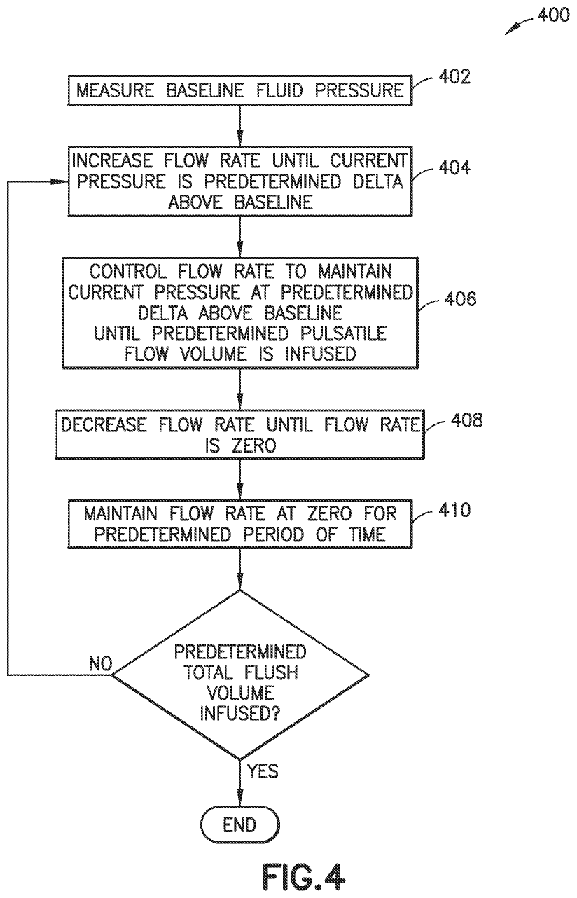

Referring now to FIG. 4, FIG. 4 is a flowchart of non-limiting embodiments or aspects of a process 400 for pulsatile flow IV line flushing. In some non-limiting embodiments or aspects, one or more of the steps of process 300 may be performed (e.g., completely, partially, etc.) by infusion pump 202 (e.g., one or more devices of a system of infusion pump 202, etc.). In some non-limiting embodiments or aspects, one or more of the steps of process 300 may be performed (e.g., completely, partially, etc.) by another device or a group of devices separate from or including infusion pump 202, such as an computing system in communication with infusion pump 202 (e.g., one or more devices of a computing system in communication with infusion pump 202).

As shown in FIG. 4, at step 402, process 400 includes measuring a baseline fluid pressure. For example, infusion pump 202 may measure a baseline fluid pressure of an IV catheter line (e.g., an IV catheter line or lumen of catheter 204, etc.) connected to infusion pump 202 and a patient. As an example, infusion pump 202 may measure the baseline fluid pressure when a flow rate of a fluid through the IV catheter line is zero. For example, infusion pump 202 may measure and store a fluid pressure value from of the IV catheter line (e.g., typically 1.5 psi, etc.), using a fluid pressure sensing device that is incorporated in infusion pump 202, when infusion pump 202 is not flowing or pumping fluid through the IV catheter line. In such an example, infusion pump 202 may be primed and connected to the IV catheter line of catheter 204 before measuring the baseline fluid pressure of the IV catheter line.

In some non-limiting embodiments or aspects, if the measured baseline fluid pressure is measured as greater than a threshold baseline fluid pressure, infusion pump 202 sets a value of the measured baseline fluid pressure as the threshold baseline fluid pressure for determining a predetermined delta pressure setting above the measured baseline fluid pressure. For example, infusion pump 202 may limit the baseline fluid pressure to a maximum safety pressure (e.g., 2.0 psi, etc.).

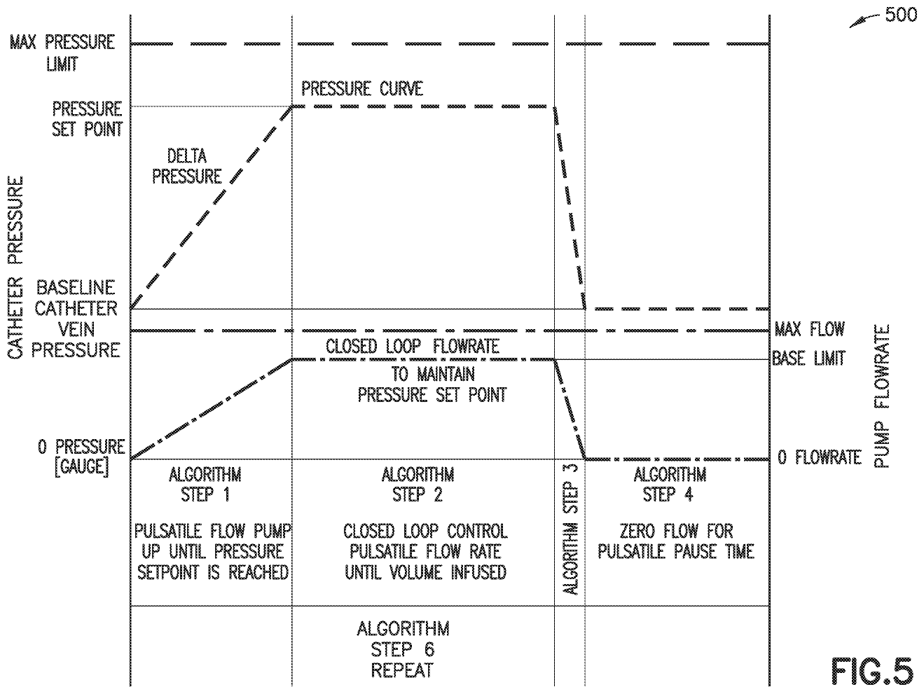

As shown in FIG. 4, at step 404, process 400 includes increasing a flow rate until a current fluid pressure is a predetermined delta above a baseline fluid pressure. For example, and referring also to FIG. 5, which is a graph 500 illustrating steps of stages of non-limiting embodiments or aspects of process 400 for pulsatile flow IV line flushing, infusion pump 202 may increase the flow rate of the fluid through the IV catheter line until a current pressure of the IV catheter line satisfies a predetermined delta pressure setting (e.g., 3.0 psi, etc.) above the measured baseline fluid pressure (e.g., a sum of the predetermined delta pressure setting and the measured baseline fluid pressure, etc.).

In some non-limiting embodiments or aspects, infusion pump 202 linearly increases the flow rate of the fluid through the IV catheter line until the current pressure of the IV catheter line satisfies the predetermined delta pressure setting above the measured baseline fluid pressure.

In some non-limiting embodiments or aspects, infusion pump 202 controls the flow rate of the fluid through the IV catheter line to remain at or below a safety threshold flow rate. For example, if the flow rate, which may be measured by a flow sensor of infusion pump 202 or an external flow sensor inline and/or attached to catheter 204, reaches a predetermined safety limit (e.g., 2 mL/s for a pulsatile flow, etc.), infusion pump 202 may not further increase the flow rate beyond the predetermined safety limit (e.g., even if the current pressure of the IV catheter line does not satisfy the predetermined delta pressure setting above the measured baseline fluid pressure, etc.).

As shown in FIG. 4, at step 406, process 400 includes controlling a flow rate to maintain a current fluid pressure at a predetermined delta above a baseline fluid pressure until a predetermined pulsatile flow volume is infused. For example, and referring also to FIG. 5, in response to the current pressure of the IV catheter line satisfying the predetermined delta pressure setting above the measured baseline fluid pressure, infusion pump 202 may control, based on the current pressure of the IV catheter line, the flow rate of the fluid through the IV catheter line to maintain the current pressure of the IV catheter line at the predetermined delta pressure setting above the baseline fluid pressure until a predetermined pulsatile flow volume (e.g., 1 ml for a pulsatile flow, etc.) is infused through the IV catheter line. As an example, infusion pump 202 may adjust the flow rate of the fluid through the IV catheter line via a closed loop control process (e.g., based on the measured flow rate and/or pressure, etc.) to maintain the current pressure in the IV catheter line at the predetermined delta pressure setting above the measured baseline fluid pressure until the predetermined pulsatile flow volume is infused through the IV catheter line.

As shown in FIG. 4, at step 408, process 400 includes decreasing a flow rate of a fluid until the flow rate is zero. For example, and referring also to FIG. 5, in response to the predetermined pulsatile flow volume being infused into the IV catheter line, infusion pump 202 may decrease the flow rate of the fluid through the IV catheter line until the flow rate is zero.

In some non-limiting embodiments or aspects, infusion pump 202 linearly decreases the flow rate of the fluid through the IV catheter line until the flow rate is zero.

As shown in FIG. 4, at step 410, process 400 includes maintaining a flow rate of a fluid at zero for a predetermined period of time. For example, and referring also to FIG. 5, in response to decreasing the flow rate of the fluid through the IV catheter line to zero, infusion pump 202 may maintain the flow rate of the fluid through the IV catheter line at zero for a predetermined period of time (e.g., 0.5 s for a pulsatile flow, etc.).

As shown in FIG. 4, at step 412, process 400 includes determining whether a predetermined total flush volume is infused. For example, infusion pump 202 may determine whether a predetermined total flush volume is infused. As an example, infusion pump 202 may determine, using the measured flow rate and time and/or a measured volume delivered, whether a predetermined total flush volume has been infused through the IV catheter line.

In response to determining in step 412 that the predetermined total flush volume is infused in step 412, process 400 may return to step 404. For example, and referring also to FIG. 5, in response to determining in step 412 that the predetermined total flush volume is infused, infusion pump 202 may return processing to step 404 and increase the flow rate of the fluid through the IV catheter line until a current pressure of the IV catheter line satisfies a predetermined delta pressure setting above the measured baseline fluid pressure.

In response to determining in step 412 that the predetermined total flush volume is infused, the process 400 for pulsatile flow IV line flushing may end. For example, in response to determining in step 412 that the predetermined total flush volume is not infused, infusion pump 202 may end the process 400 for pulsatile flow IV line flushing.

Although embodiments or aspects have been described in detail for the purpose of illustration and description, it is to be understood that such detail is solely for that purpose and that embodiments or aspects are not limited to the disclosed embodiments or aspects, but, on the contrary, are intended to cover modifications and equivalent arrangements that are within the spirit and scope of the appended claims. For example, it is to be understood that the present disclosure contemplates that, to the extent possible, one or more features of any embodiment or aspect can be combined with one or more features of any other embodiment or aspect. In fact, any of these features can be combined in ways not specifically recited in the claims and/or disclosed in the specification. Although each dependent claim listed below may directly depend on only one claim, the disclosure of possible implementations includes each dependent claim in combination with every other claim in the claim set.

1. An actuator actuated valve for maintaining multi-lumen patency, comprising:
a valve manifold including an input port, a first output port, and a second output port; and
a valve spool extending within the valve manifold, wherein the valve spool defines a first valve passageway and a second valve passageway, wherein the valve spool is rotatable relative to the valve manifold between a first position in which the input port and the first output port are in fluid communication via the first valve passageway and a second position in which the input port and the second output port are in fluid communication via the second valve passageway.

2. The actuator actuated valve of claim 1, wherein, when the valve spool is between the first position and the second position, the input port is not in fluid communication with the first output port and the second output port.

3. The actuator actuated valve of claim 1, wherein, when the valve spool is between the first position and the second position, the input port is in fluid communication with each of the first output port and the second output port.

4. The actuator actuated valve of claim 1, wherein the valve spool is rotatable relative to the valve manifold between the first position, the second position, and a third position in which the input port is not in fluid communication with the first output port and the second output port.

5. The actuator actuated valve of claim 1, wherein, when the valve spool is in the first position, the input port is not in fluid communication with the second output port, and wherein, when the valve spool is in the second position, the input port is not in fluid communication with the first output port.

6. The actuator actuated valve of claim 1, wherein the valve manifold further includes at least one further output port, wherein the valve spool further defines at least one further valve passageway, wherein the valve spool is further rotatable relative to the valve manifold between the first position, the second position, and at least one further position in which in the input port and the at least one further output port are in fluid communication via the at least one further valve passageway.

7. The actuator actuated valve of claim 6, wherein the valve spool is rotatable between a plurality of positions including the first position, the second position, and the at least one further position, and wherein, in the plurality of positions, (i) a single output port of the first output port, the second output port, and the at least one further output port is in fluid communication with the input port via a single valve passageway of the first valve passageway, the second valve passageway, and the at least one further valve passageway and (ii) the other output ports of the first output port, the second output port, and the at least one further output port are not in fluid communication with the input port via the other valve passageways of the first valve passageway, the second valve passageway, and the at least one further valve passageway.

8. The actuator actuated valve of claim 1, further comprising:
a rotary actuator configured to rotate the valve spool relative to the valve manifold between the first position and the second position.

9. The actuator actuated valve of claim 8, further comprising:
a communication device configured to receive control data from an external computing device; and
a processor configured to control, based on the control data, the rotary actuator to rotate the valve spool relative to the valve manifold between the first position and the second position.

10. The actuator actuated valve of claim 9, wherein the communication device includes a wireless communication device configured to wirelessly receive the control data from the external device.

11. The actuator actuated valve of claim 9, further comprising:
a housing connected to the valve spool, wherein the housing includes the rotary actuator, the communication device, and the processor.

12. The actuator actuated valve of claim 11, wherein the housing further includes a memory, a power source, and a user feedback device.

13. The actuator actuated valve of claim 11, further comprising:
a first check valve integrated with the first output port;
a second check valve integrated with the second output port.

14. The actuator actuated valve of claim 1, wherein the input port is removably connected to a fluid output of a fluid reservoir.

15. The actuator actuated valve of claim 1, wherein the input port is integrated with a fluid output of a fluid reservoir.

16. The actuator actuated valve of claim 15, wherein the fluid reservoir includes a syringe, and wherein the input port is integrated with a tip of the syringe.

17. The actuator actuated valve of claim 1, wherein the valve spool includes an actuator interface extending from an end of the valve spool, and wherein the actuator interface is configured to interface with a rotary actuator.

18. A system for maintaining multi-lumen patency, comprising:
an actuator actuated diverter valve including:
a valve manifold including an input port, a first output port, and a second output port; and
a valve spool extending within the valve manifold, wherein the valve spool defines a first valve passageway and a second valve passageway, wherein the valve spool is rotatable relative to the valve manifold between a first position in which the input port and the first output port are in fluid communication via the first valve passageway and a second position in which the input port and the second output port are in fluid communication via the second valve passageway; and
a rotary actuator configured to rotate the valve spool relative to the valve manifold between the first position and the second position;
a multi-lumen catheter including:
a first lumen connected to the first output port; and
a second lumen connected to the second output port; and
a fluid reservoir connected to the input port.

19. The system of claim 18, wherein the fluid reservoir includes one of an IV bag, an infusion pump, and a syringe.

20. The system of claim 18, further comprising:
a communication device configured to receive control data from an external computing device; and
a processor configured to control, based on the control data, the rotary actuator to rotate the valve spool relative to the valve manifold between the first position and the second position.

21.-66. (canceled)
